(19) United States
(12) Patent Application Publication
(10) Pub. No.: US 2012/0135947 A1
(43) Pub. Date: May 31, 2012

(54) OIL-IN-WATER EMULSION COMPOSITION CONTAINING DIFLUPREDNATE AND TOBRAMYCIN

(75) Inventor: Yuko SHIKAMURA, Hyogo (JP)

(73) Assignees: MITSUBISHI TANABE PHARMA CORPORATION, Osaka (JP); SENJU PHARMACEUTICAL CO., LTD., Osaka (JP)

(21) Appl. No.: 13/304,988

(22) Filed: Nov. 28, 2011

Related U.S. Application Data
(60) Provisional application No. 61/417,651, filed on Nov. 29, 2010.

(51) Int. Cl. A61K 31/7036; A61K 8/60; A61P 27/16; A61Q 19/00; A61P 27/02; A61P 11/02

(52) U.S. Cl. 514/40

(57) ABSTRACT

The present invention provides an oil-in-water emulsion composition for topical administration, containing (a) tobramycin, (b) difluprednate, (c) water, (d) oil and (e) an emulsifier. Moreover, it provides a method for stabilizing tobramycin, which includes mixing (a) tobramycin, (b) difluprednate, (c) water, (d) oil and (e) an emulsifier to form an oil-in-water emulsion. The present invention can provide an oil-in-water emulsion composition containing tobramycin, which can maintain tobramycin content stably even when a non-ionic surfactant is added.

### TECHNICAL FIELD OF THE INVENTION

The present invention relates to a composition containing difluprednate and tobramycin, which shows good storage stability.

### BACKGROUND OF THE INVENTION

A combination agent of an anti-inflammatory steroid and an antibiotic is useful for inflammatory diseases caused by infections, and there are many reports of such combination agents (1)-(4). Tobramycin is an aminoglycoside antibiotic having a wide antibacterial spectrum and is often combined with steroids (2)-(4). Many of steroids are hardly water-soluble, and are generally developed as suspensions or ointments. As an additive necessary for suspensions or ointments, non-ionic surfactants such as polysorbate 80, tyloxapol and the like are widely used as suspending agents or dispersing agents. As for the combination agents of steroid and tobramycin, however, the background art is silent on the storage stability of tobramycin in the presence of a non-ionic surfactant in aqueous solutions. Difluprednate is a hardly water-soluble steroid showing a superior anti-inflammatory action and a superior antiallergic action (5)-(6). Since difluprednate dissolves in oils such as castor oil and the like, it can be prepared as an oil-in-water emulsion which is a preferable dosage form from the aspect of tissue transferability (7). Oil-in-water emulsions also contain a non-ionic surfactant as an emulsifier (7).

### REFERENCES

1. JP-A-2001-089378 (U.S. Pat. No. 6,277,829)
2. JP-A-2007-509952 (US 2005/0197303)
3. JP-A-08-231405 (U.S. Pat. No. 5,149,693)
4. JP-A-07-309763 (EP 661055)
5. JP-B-45-28370 (U.S. Pat. No. 3,780,177)
6. JP-B-45-28371 (U.S. Pat. No. 3,784,692)
7. JP-A-11-29483 (U.S. Pat. No. 6,114,319)

### SUMMARY OF THE INVENTION

The present invention aims to provide a composition for topical administration, which contains difluprednate and tobramycin, and shows good storage stability.

The present inventor has found that the stability of difluprednate and tobramycin can be improved by forming an aqueous composition of difluprednate and tobramycin as an oil-in-water emulsion by adding an oil and an emulsifier, which resulted in the completion of the present invention.

Accordingly, the present invention provides the following.
(1) An oil-in-water emulsion composition for topical administration, comprising (a) tobramycin, (b) difluprednate, (c) water, (d) oil and (e) an emulsifier.
(2) The composition described in (1), wherein said oil is selected from the group consisting of castor oil, peanut oil, cottonseed oil, soybean oil, olive oil and medium-chain triglyceride.
(3) The composition described in (2), wherein said oil is castor oil.
(4) The composition described in any one of (1) to (3), wherein said emulsifier is a non-ionic surfactant.
(5) The composition described in (4), wherein said non-ionic surfactant is at least one selected from the group consisting of polysorbate 80, tyloxapol, polyoxyethylene hydrogenated castor oil 60 and polyoxyl 40 stearate.
(6) The composition described in any one of (1) to (5), further comprising at least one selected from the group consisting of boric acid, concentrated glycerol and sodium chloride as a tonicity agent.
(7) The composition described in any one of (1) to (6), which is an eye drop, a nasal drop, an ear drop or a lotion.
(8) The composition described in (1), which comprises 0.01-0.2 (w/v)% of difluprednate, 0.1-1 (w/v)% of tobramycin, 1-20 (w/v)% of castor oil and 1-20 (w/v)% of polysorbate 80, and has a pH of 4-7.
(9) A method for stabilizing tobramycin, comprising mixing (a) tobramycin, (b) difluprednate, (c) water, (d) oil and (e) an emulsifier to form an oil-in-water emulsion.

For preparation of a liquid containing tobramycin and a hardly water-soluble drug, addition of a surfactant and/or the like to be used as a suspending agent or an emulsifier is necessary. Tobramycin has been found to show decreased storage stability in the presence of a non-ionic surfactant. According to the present invention, however, an oil-in-water emulsion composition showing good storage stability of tobramycin can be provided.

### DETAILED DESCRIPTION OF THE INVENTION

The present invention provides an oil-in-water emulsion composition comprising difluprednate, tobramycin, oil, water, and an emulsifier (hereinafter to be referred to as the composition of the present invention).

Difluprednate (6α,9α-difluoroprednisolone 17-butyrate 21-acetate) which can be used for the composition of the present invention is a steroidal anti-inflammatory drug known to show a superior anti-inflammatory action and a superior antiallergic action by transdermal administration or ocular instillation administration.

Tobramycin which can be used for the composition of the present invention is an aminoglycoside antibiotic known to show an antibacterial action (bacteriocidal action) by inhibiting protein synthesis in bacteria.

Oils that can be used for the composition of the present invention may be any as long as they are low toxic, low irritative and applicable to the eye. Preferable examples include those containing fatty acid esters of glycerol, such as castor oil, peanut oil, cottonseed oil, soybean oil, olive oil, medium-chain triglyceride [e.g., Miglyol (trade name, Mitsuba Trading Co., Ltd.)] and the like. More preferred are, for example, castor oil, medium-chain triglyceride (e.g., Miglyol) and the like, which can dissolve difluprednate well, and particularly preferred is castor oil.

Water that can be used for the composition of the present invention is not particularly limited as long as it is generally added to pharmaceutical compositions, and purified water, distilled water for injection and the like can be mentioned.

As the kind of emulsifier that can be used for the composition of the present invention, non-ionic surfactant and the like can be mentioned. Examples thereof include polyoxyethylene sorbitan fatty acid esters, polyoxyethylene hydrogenated castor oils, alkyl aryl polyether alcohol type polymers, polyoxyethylene fatty acid esters, polyoxyethylene polyoxypropylene glycols and sucrose fatty acid esters. Preferred are polyoxyethylene sorbitan monooleate, polyoxyethylene sorbitan monolaurate, polyoxyethylene sorbitan monopalmitate, polyoxyethylene sorbitan monostearate, polyoxyethylene sorbitan tristearate, polyoxyethylene hydrogenated castor oil 10, polyoxyethylene hydrogenated castor oil 40, polyoxyethylene hydrogenated castor oil 50, polyoxyethylene hydrogenated castor oil 60, tyloxapol, polyoxyl stearate and the like, and particularly preferred are polysorbate 80, polyoxyethylene hydrogenated castor oil 60, tyloxapol and polyoxyl 40 stearate. These may be used in combination.

While the amount of difluprednate and tobramycin to be contained in the composition of the present invention is not particularly limited, for example, difluprednate is contained in the composition in a proportion of not less than 0.001 (w/v)%, preferably not less than 0.005 (w/v)%, more preferably not less than 0.01 (w/v)%, not more than 0.4 (w/v)%, preferably not more than 0.3 (w/v)%, more preferably not more than 0.2 (w/v)%. The proportion of tobramycin in the composition is not less than 0.01 (w/v)%, preferably not less than 0.05 (w/v)%, more preferably not less than 0.1 (w/v)%, not more than 10 (w/v)%, preferably not more than 5 (w/v)%, more preferably not more than 1 (w/v)%.

The amount of oil to be contained in the composition of the present invention is not particularly limited as long as it can generally provide an oil-in-water emulsion. The amount of oil in the composition is not less than 0.1 (w/v)%, preferably not less than 0.5 (w/v)%, more preferably not less than 1 (w/v)%, not more than 40 (w/v)%, preferably not more than 30 (w/v)%, more preferably not more than 20 (w/v)%.

The amount of water to be contained in the composition of the present invention is not particularly limited, it is not less than 20 (w/v)%, preferably not less than 50 (w/v)%, more preferably not less than 60 (w/v)%, not more than 99.8 (w/v)%, preferably not more than 99 (w/v)%, more preferably not more than 98 (w/v)%, of the composition.

The amount of an emulsifier to be contained in the composition of the present invention is not particularly limited as long as it can generally provide an oil-in-water emulsion. The content ratio thereof in the composition is not less than 0.1 (w/v)%, preferably not less than 0.5 (w/v)%, more preferably not less than 1 (w/v)%, not more than 40 (w/v)%, preferably not more than 30 (w/v)%, more preferably not more than 20 (w/v)%.

While the combination of the amounts of the above-mentioned components in the composition of the present invention is not particularly limited, a combination of, for example, 0.001-0.4 (w/v)% of difluprednate, 0.01-10 (w/v)% of tobramycin, 0.1-40 (w/v)% of oil, 20-99.8 (w/v)% of water and 0.1-40 (w/v)% of an emulsifier, preferably 0.005-0.3 (w/v)% of difluprednate, 0.05-5 (w/v)% of tobramycin, 0.5-30 (w/v)% of oil, 50-99 (w/v)% of water and 0.5-30 (w/v)% of an emulsifier, in the composition can be mentioned. Particularly preferred is a composition of 0.01-0.2 (w/v)% of difluprednate, 0.1-1 (w/v)% of tobramycin, 1-20 (w/v)% of oil, 60-98 (w/v)% of water and 1-20 (w/v)% of an emulsifier.

The composition of the present invention can contain a water-soluble polymer to increase the stability of emulsion particles. Examples of the water-soluble polymer include povidone (polyvinylpyrrolidone), polyvinyl alcohol, hydroxyethylcellulose, hydroxypropylcellulose, methylcellulose, hydroxypropylmethylcellulose, carboxymethylcellulose, and salts thereof and the like. The water-soluble polymer can be added in about 0.001- about 3 (w/v)% to the composition.

The composition of the present invention can contain a tonicity agent. Examples of the tonicity agent include boric acid, sodium chloride, concentrated glycerol, potassium chloride, D-mannitol and the like. The above-mentioned tonicity agent can be added as long as the storage stability of difluprednate and tobramycin is not markedly decreased. Particularly, the tonicity agent is preferably at least one selected from the group consisting of boric acid, concentrated glycerol and sodium chloride, which do not easily influence the storage stability of difluprednate and tobramycin. These may be used in combination.

When boric acid is added to the composition of the present invention, the concentration thereof is not less than 0.001 (w/v)%, preferably not less than 0.01 (w/v)%, more preferably not less than 0.05 (w/v)%, and not more than 3.3 (w/v)%, preferably not more than 2.0 (w/v)%, more preferably not more than 1.8 (w/v)%, of the composition. When sodium chloride is added, the concentration thereof is not less than 0.001 (w/v)%, preferably not less than 0.01 (w/v)%, more preferably not less than 0.05 (w/v)%, and not more than 1.6 (w/v)%, preferably not more than 0.9 (w/v)%, more preferably not more than 0.8 (w/v)%, of the composition. When concentrated glycerol is added, the concentration thereof is not less than 0.001 (w/v)%, preferably not less than 0.01 (w/v)%, more preferably not less than 0.05 (w/v)%, and not more than 2.6 (w/v)%, preferably not more than 2.2 (w/v)%, more preferably not more than 2.0 (w/v)%, of the composition.

The composition of the present invention is adjusted to have an osmotic pressure of about 150- about 1100 mOsm, preferably about 150- about 650 mOsm, more preferably about 220- about 480 mOsm, by the addition of a tonicity agent.

The composition of the present invention can contain a buffering agent. Examples of the buffering agent include acetate salts such as sodium acetate and the like, phosphate salts such as monosodium dihydrogen phosphate, disodium monohydrogen phosphate, monopotassium dihydrogen phosphate, dipotassium monohydrogen phosphate and the like, amino acid salts such as ε-aminocaproic acid, sodium glutamate and the like, citric acid and a salt thereof, tromethamol, 4-(2-hydroxyethyl)-1-piperazineethanesulfonic acid and the like.

The buffering agent to be added to the composition of the present invention can be added as long as it does not decrease the storage stability of difluprednate and tobramycin. The buffering agent can be added in about 0.01- about 2 (w/v)% of the composition.

The composition of the present invention can contain a preservative as long as it does not markedly decrease the storage stability of difluprednate and tobramycin. Examples of the preservative include p-hydroxybenzoate ester such as methyl p-hydroxybenzoate, propyl p-hydroxybenzoate and the like, alcohol compounds such as chlorobutanol, benzyl alcohol and the like, sodium dehydroacetate, thimerosal, chlorite and the like.

The composition of the present invention can additionally contain various additives such as stabilizer, antioxidant, chelating agent, pH adjusting agent, thickener and the like. Examples of the antioxidant include ascorbic acid and a salt thereof, tocopherol, sodium thiosulfate, sodium hydrogen sulfite, pyruvic acid and a salt thereof and the like. Examples of the chelating agent include sodium edetate, citric acid and a salt thereof and the like. Examples of the pH adjusting agent include hydrochloric acid, phosphoric acid, acetic acid, sulfuric acid, boric acid, borax, sodium hydroxide, potassium hydroxide, sodium carbonate, sodium hydrogen carbonate, aqueous ammonia and the like. Particularly, examples of the acidic pH adjusting agent include hydrochloric acid, phosphoric acid, acetic acid, sulfuric acid and boric acid.

The composition of the present invention can be provided as an aqueous preparation such as an oil-in-water (O/W) emulsion, a microemulsion and the like.

The mean particle size (median size) of an oil drop of the composition of the present invention is preferably 10-2000 nm, more preferably 20-1000 nm, particularly preferably 20-500 nm. The average particle size can be measured by a particle size distribution measuring apparatus.

The composition of the present invention preferably has pH 3-8, more preferably pH 4-7. The stability of difluprednate and tobramycin is most preferable in this pH range.

The composition of the present invention can contain other hardly water-soluble steroidal anti-inflammatory agent instead of difluprednate, as long as the storage stability of tobramycin does not decrease. Tobramycin used for the composition of the present invention may be replaced with quinolone antibiotics such as moxifloxacin and the like.

The composition of the present invention is prepared by preparing an aqueous phase containing an emulsifier and an oil phase containing difluprednate, and mixing and emulsifying these phases.

Tobramycin may be dissolved in an aqueous phase or added to an emulsion after emulsification. For uniform emulsification, a known means such as a homomixer, a homogenizer, a high-pressure homogenizer, an ultra high-pressure homogenizer (microfluidizer) and the like can be used. Other additives such as tonicity agent, buffering agent, preservative and the like may also be dissolved in an aqueous phase of an emulsifier or added to an emulsion after emulsification.

Particularly, it is desirably produced by a step of preparing a tobramycin aqueous phase by adding tobramycin, an emulsifier, a tonicity agent and a buffering agent to water, step of adjusting the pH of the tobramycin aqueous phase to 5.4-5.6 by adding a pH adjusting agent, a step of preparing a difluprednate oil phase by dissolving difluprednate in oil, a step of preparing a coarse emulsion by mixing the tobramycin aqueous phase with pH 5.4-5.6 and the difluprednate oil phase, and coarsely emulsifying the mixture by a homogenizer and the like, and a step of preparing an oil-in-water emulsion by micronizing the coarse emulsion with pH 5.4-5.6 by a homogenizer.

In the present specification, the “emulsification” refers to processing an oil phase into a number of ultrafine droplets and dispersing and maintaining them in an aqueous phase. The “coarse emulsification” refers to one form of emulsification, wherein an oil phase is processed into fine droplets of a certain level and dispersed and maintained in an aqueous phase. In this case, the size of the droplet is not uniform. The “micronization” refers to one form of emulsification, wherein a coarse emulsion is further processed using a device such as a microfluidizer and the like to further micronize the droplets of the oil phase to have a size uniform to some extent.

In the preparation step of the composition of the present invention, tobramycin, and additives such as a tonicity agent, a buffering agent and the like may be dissolved in an aqueous phase or added to an emulsion after emulsification.

The composition of the present invention preferably contains respective components at proportions of 0.01-0.2 (w/v)% of difluprednate, 0.1-1 (w/v)% of tobramycin, 1-20 (w/v)% of castor oil, and 1-20 (w/v)% of polysorbate 80, and has pH 4-7.

The most preferable composition of the present invention contains respective components at proportions of 0.05 (w/v)% of difluprednate, 0.3 (w/v)% of tobramycin, 5.0 (w/v)% of castor oil, and 4.0 (w/v)% of polysorbate 80, and has a pH of about 5.5.

The composition of the present invention is preferably used as a preparation for topical administration to the eye, nose, ear or skin, and as an eye drop, a nasal drop, an ear drop or a lotion.

The composition of the present invention has a superior anti-inflammatory action, an antiallergic action and an antibacterial action. Accordingly, in ophthalmologic diseases, the composition is useful for the prophylaxis or treatment of various inflammatory diseases or allergic diseases such as allergic conjunctivitis, spring catarrh, marginal blepharitis, catarrhal conjunctivitis, uveitis and the like. In otorhinolaryngologic diseases, the composition is useful for the prophylaxis or treatment of inflammatory diseases or allergic diseases in external ear, middle ear (including auditory tube) or upper airway (external otitis, middle otitis, allergic rhinitis etc.) or postoperative care. In addition, the composition can be also advantageously used for topical administration to eye, nose, ear, skin and the like.

The composition of the present invention can be safely administered to mammal (human, dog, rabbit, bovine, horse, monkey, cat, sheep etc.)

While the dose of the composition of the present invention varies depending on the kind and symptom of the disease, the age and body weight of the patients and the like, when it is used, for example, as an eye drop for an adult, an eye drop containing 0.01-0.2 (w/v)% of difluprednate and 0.1-1.0 (w/v)% of tobramycin is desirably instilled by 1-2 drops/dose per one eye of a patient about 2 to 4 times per day according to the symptoms.

In addition, the present invention provides a method for stabilizing tobramycin, comprising mixing (a) tobramycin, (b) difluprednate, (c) water, (d) oil and (e) an emulsifier to form an oil-in-water emulsion (hereinafter to be referred to as the method of the present invention).

Difluprednate and tobramycin that can be used for the stabilizing method of the present invention are as described above for the composition of the present invention.

Examples of the kind of the oil usable for the method of the present invention include castor oil, peanut oil, cottonseed oil, soybean oil, olive oil, medium-chain triglyceride [e.g., Miglyol (trade name, Mitsuba Trading Co., Ltd.)] and the like as mentioned above. More preferred are castor oil, medium-chain triglyceride (e.g., Miglyol) and the like showing high solubility of difluprednate, and particularly preferred is castor oil.

As the kind of emulsifier that can be used for the method of the present invention, the aforementioned non-ionic surfactant and the like can be mentioned. Examples thereof include polyoxyethylene sorbitan fatty acid esters, polyoxyethylene hydrogenated castor oils, alkyl aryl polyether alcohol type polymers, polyoxyethylene fatty acid esters, polyoxyethylene polyoxypropylene glycols or sucrose fatty acid esters, preferably polyoxyethylene sorbitan monooleate, polyoxyethylene sorbitan monolaurate, polyoxyethylene sorbitan monopalmitate, polyoxyethylene sorbitan monostearate, polyoxyethylene sorbitan tristearate, polyoxyethylene hydrogenated castor oil 10, polyoxyethylene hydrogenated castor oil 40, polyoxyethylene hydrogenated castor oil 50, polyoxyethylene hydrogenated castor oil 60, tyloxapol, polyoxyl stearates and the like. Particularly preferred are polysorbate 80, polyoxyethylene hydrogenated castor oil 60, tyloxapol and polyoxyl 40 stearate. These may be used in combination.

The aforementioned various additives such as tonicity agent, buffering agent, preservative, stabilizer, antioxidant, chelating agent, pH adjusting agent, thickener and the like may be used for the method of the present invention.

As the tonicity agent that can be used for the method of the present invention, at least one selected from the group consisting of boric acid, sodium chloride and concentrated glycerol, which do not easily influence the storage stability of difluprednate and tobramycin, as mentioned above, is preferable. These may be used in combination.

When boric acid is added, the concentration thereof is not less than 0.001 (w/v)%, preferably not less than 0.01 (w/v)%, more preferably not less than 0.05 (w/v)%, and not more than 3.3 (w/v)%, preferably not more than 2.0 (w/v)%, more preferably not more than 1.8 (w/v)%, of the composition. When sodium chloride is added, the concentration thereof is not less than 0.001 (w/v)%, preferably not less than 0.01 (w/v)%, more preferably not less than 0.05 (w/v)%, and not more than 1.6 (w/v)%, preferably not more than 0.9 (w/v)%, more preferably not more than 0.8 (w/v)%, of the composition. When concentrated glycerol is added, the concentration thereof is not less than 0.001 (w/v)%, preferably not less than 0.01 (w/v)%, more preferably not less than 0.05 (w/v)%, and not more than 2.6 (w/v)%, preferably not more than 2.2 (w/v)%, more preferably not more than 2.0 (w/v)%, of the composition.

The present invention is explained in more detail in the following by referring to Examples. It is needless to say that the present invention is not limited by the Examples.

### EXAMPLES

### Experimental Example 1

An aqueous tobramycin solution, a suspension of tobramycin and difluprednate, and an emulsion of an aqueous tobramycin solution and difluprednate dissolved in castor oil, each containing various additives, were prepared, and the stability of tobramycin was compared.

### Test Operation

TABLE 1
Formulations of Examples 1 to 3 and Comparative Examples 1 to 5

| component | | Ex. | Ex. | Ex. | Comp. | Comp. | Comp. | Comp. | Comp. |
|---|---|---|---|---|---|---|---|---|---|
| (g/100 mL) | grade | 1 | 2 | 3 | Ex. 1 | Ex. 2 | Ex. 3 | Ex. 4 | Ex. 5 |
| difluprednate | JPC | 0.05 | 0.05 | 0.05 | — | — | — | — | 0.05 |
| tobramycin | EP | 0.3 | 0.3 | 0.3 | 0.3 | 0.3 | 0.3 | 0.3 | 0.3 |
| castor oil | JP | 5.0 | 5.0 | 5.0 | — | — | — | — | — |
| sodium acetate | JP | 0.05 | 0.05 | 0.05 | 0.05 | 0.05 | 0.05 | 0.05 | 0.05 |
| polysorbate 80 | JP | 4.0 | 4.0 | 4.0 | 4.0 | 4.0 | 4.0 | — | 4.0 |
| boric acid | JP | — | 1.2 | — | — | 1.0 | 0.1 | — | — |
| concentrated | JP | — | — | 1.7 | — | — | 1.6 | — | — |
| glycerol | | | | | | | | | |
| sodium edetate | JP | — | 0.02 | 0.02 | — | 0.02 | 0.02 | — | — |
| sodium hydroxide/ | JP | q.s. | q.s. | q.s. | q.s. | q.s. | q.s. | q.s. | q.s. |
| hydrochloric acid | | | | | | | | | |
| purified water | — | q.s. | q.s. | q.s. | q.s. | q.s. | q.s. | q.s. | q.s. |
| (total amount | | | | | | | | | |
| 100 mL) | | | | | | | | | |
| pH | — | 5.5 | 5.5 | 5.5 | 5.5 | 5.5 | 5.5 | 5.5 | 5.5 |

Examples 1 to 3 are emulsions, Comparative Examples 1 to 4 are aqueous solutions, and Comparative Example 5 is a suspension.

In Tables 1 to 5, the grade of JP means the Japanese Pharmacopoeia, EP means the European Pharmacopoeia, and JPC means the Japanese Pharmaceutical Codex.
q.s. means quantum sufficit.

### Preparation Method of Comparative Examples 1 to 5

1) Based on the above-mentioned formulation, sodium acetate (manufactured by Wako Pure Chemical Industries, Ltd.), polysorbate 80 (manufactured by NOF CORPORATION), concentrated glycerol (manufactured by Sakamoto Yakuhin Kogyo Co., Ltd.), boric acid (manufactured by Wako Pure Chemical Industries, Ltd.), sodium edetate (manufactured by NACALAI TESQUE, INC. or manufactured by KANTO CHEMICAL CO., INC.) and tobramycin (manufactured by Teva Pharmaceutical Industries Ltd.) were dissolved in purified water.
2) Sodium hydroxide reagent (manufactured by NACALAI TESQUE, INC.) or hydrochloric acid (manufactured by NACALAI TESQUE, INC.) was added to adjust the pH to 5.4-5.6.
3) Purified water was added to dilute to a predetermined volume.

In Comparative Example 5, difluprednate (manufactured by Mitsubishi Tanabe Pharma Corporation) was further added to give a difluprednate suspension.

### Preparation Method of Example 1

1) Based on the above-mentioned formulation, polysorbate 80 and sodium acetate were dissolved in purified water.
2) Hydrochloric acid was added to adjust the pH to 5.4-5.6.
3) Separately, difluprednate was added to castor oil (manufactured by NOF CORPORATION), and dissolved in a water bath (85-95° C.) (solution A).
4) Solution A was gradually added to the solution of 2) (65-75° C.) to allow coarse emulsification (stirrer: Robo Mics, TOKUSHU KIKA KOGYO, rotating speed: about 8000 rpm, stirring time: 1 hr after addition of solution A).
5) After allowing to cool to room temperature, pH was confirmed (pH 5.4-5.6).
6) Purified water was added to dilute to 50% of a predetermined volume.
7) Using a high-pressure emulsifier (microfluidizer, Microfluidics Corporation), a micronized emulsion was prepared at treatment pressure about 1500 kgf/cm², water bath temperature for sample cooler about 35-45° C., number of passes 20 times.
8) Tobramycin was added and dissolved in the micronized emulsion.
9) Hydrochloric acid was added to the micronized emulsion to adjust the pH to 5.4-5.6.
10) Purified water was added to dilute to a predetermined volume.

### Preparation Method of Example 2

1) Based on the above-mentioned formulation, polysorbate 80, sodium acetate, sodium edetate, boric acid and tobramycin were dissolved in purified water.
2) Separately, difluprednate was added to castor oil, and dissolved in a water bath (85-95° C.) (solution A).
3) Solution A was gradually added to the solution of 1) (65-75° C.) to allow coarse emulsification (stirrer: Robo Mics, TOKUSHU KIKA KOGYO, rotating speed: about 8000 rpm, stirring time: 1 hr after addition of solution A).
4) After allowing to cool to room temperature, hydrochloric acid was added to adjust the pH to 5.4-5.6.
5) Purified water was added to dilute to a predetermined volume, and pH was confirmed (pH 5.4-5.6).
6) Using a high-pressure emulsifier (microfluidizer, Microfluidics Corporation), a micronized emulsion was prepared at treatment pressure about 1500 kgf/cm², water bath temperature for sample cooler about 35-45° C., number of passes 20 times.

### Preparation Method of Example 3

1) Based on the above-mentioned formulation, polysorbate 80, sodium acetate, sodium edetate and tobramycin were dissolved in purified water.
2) Hydrochloric acid was added to adjust the pH to 5.4-5.6.
3) Separately, difluprednate was added to castor oil, and dissolved in a water bath (85-95° C.) (solution A).
4) Solution A was gradually added to the solution of 2) (65-75° C.) to allow coarse emulsification (stirrer: Robo Mics, TOKUSHU KIKA KOGYO, rotating speed: about 8000 rpm, stirring time: 1 hr after addition of solution A).
5) After allowing to cool to room temperature, pH was confirmed (pH 5.4-5.6).
6) Purified water was added to dilute to a predetermined volume.
7) Using a high-pressure emulsifier (microfluidizer, Microfluidics Corporation), a micronized emulsion was prepared at treatment pressure about 1500 kgf/cm², water bath temperature for sample cooler about 35-45° C., number of passes 20 times.
8) Concentrated glycerol was added and dissolved in the micronized emulsion, and pH was confirmed (pH 5.4-5.6).

### Test Conditions

Examples 1 to 3 and Comparative Examples 1 to 5 prepared above were filled by 5 mL in 5 mL colorless glass ampoules and used as samples. The tobramycin content in the samples on preparation and after storage at 60° C. for 2 weeks was measured under the following conditions. The residual ratio (%) of tobramycin content after storage at 60° C. for 2 weeks to that on preparation as 100% was determined. In Examples 1 to 3, the difluprednate content of the samples on preparation and after storage at 60° C. for 2 weeks was measured under the following conditions, and the residual ratio (%) after storage at 60° C. for 2 weeks was determined. In Examples 1 to 3, moreover, the osmotic pressure on preparation was measured under the following conditions.

### 1. Tobramycin Content

Tobramycin was quantified by the following method.

### 1) Preparation of Sample Solution and Standard Solution

(1) Sample Solution and Standard Solution before Derivatization

A sample (2 mL) was accurately measured, purified water was precisely added thereto to 50 mL and the mixture was used as a sample solution before derivatization. Separately, tobramycin (0.033 g) was precisely measured, water (20 mL) and 1 N sulfuric acid (1 mL) were added to dissolve tobramycin, and purified water was accurately added to 50 mL. This solution (10 mL) was accurately measured, purified water was accurately added thereto to 50 mL, and the mixture was used as a standard solution before derivatization.

### (2) Derivatization Operation

5 mL each of the sample solution before derivatization and standard solution before derivatization was accurately measured and placed in a 50 mL measuring flask. A 2,4-dinitrofluorobenzene test solution (10 mL) and a tris(hydroxymethyl)aminomethane test solution (10 mL) were respectively added, the mixtures were shaken and the flasks were capped. These measuring flasks were placed in a water bath (about 60° C.) and heated for about 50 min. The measuring flasks were taken out from the water bath, and allowed to cool for 10 min. Acetonitrile was accurately added to 50 mL, and the mixtures were used as a sample solution and a standard solution, respectively.

### 2) Preparation of Test Solution

1 N Sulfuric acid

Sulfuric acid (30 mL) was gradually added to purified water (1000 mL) with stirring, and the mixture was allowed to cool.

2,4-Dinitrofluorobenzene test solution

2,4-Dinitrofluorobenzene was dissolved in ethyl alcohol to 10 mg/mL.

Tris(hydroxymethyl)aminomethane test solution

Tris(hydroxymethyl)aminomethane was dissolved in water to 15 mg/mL. This solution (40 mL) was diluted with dimethyl sulfoxide (DMSO) to 200 mL.

### 3) Measurement Conditions

The sample solution and standard solution (20 μL) prepared in 1) were subjected to the measurement by a liquid chromatography (HPLC) method under the following conditions.
Constitution of HPLC system
HPLC system: high performance liquid chromatography system (Shimadzu Corporation)
Solvent delivery unit: LC-10ADvp, LC-10AD
Detector: ultraviolet visible spectrophotometer (SPD-10AVvp, SPD-10A, SPD-10AV)
Autosampler: SIL-20AC, SIL-10A
Column oven: CTO-10ACvp, CTO-10AC
System controller: SCL-10Avp, CBM-20^{a}
HPLC measurement conditions
Detector: ultraviolet absorption spectrophotometer (measurement wavelength: 365 nm)
Column: commercially available column (YMC-Pack ODS-A A-302, manufactured by YMC) which is a stainless tube (inner diameter 4.6 mm, length 150 mm) filled with 5 μm of octadecylsilyl silica gel for liquid chromatography
Column temperature: constant temperature near 40° C.
Mobile phase: Tris(hydroxymethyl)aminomethane (2.0 g) was dissolved in purified water (800 mL). To this solution was added 1 N sulfuric acid (20 mL), and acetonitrile was added to 2000 mL.
Flow rate: 1.2 mL/min

### 2. Difluprednate Content

The sample (2 mL) was accurately measured, and purified water was precisely added thereto to 50 mL. This solution (2 mL) was accurately measured, acetonitrile was accurately added thereto to 20 mL, and the mixture was used as a sample solution. Separately, difluprednate (about 0.02 g) was accurately measured, and acetonitrile was added to dissolve difluprednate to give an accurate amount of 100 mL. This solution (2 mL) was accurately measured, and acetonitrile was added to 20 mL. This solution (2 mL) was accurately measured, and acetonitrile was added to 20 mL, and the mixture was used as a standard solution. The sample solution and standard solution (20 mL) were subjected to the measurement by a liquid chromatography (HPLC) method under the following conditions.
Constitution of HPLC system
HPLC system: high performance liquid chromatography system (Shimadzu Corporation)
Solvent delivery unit: LC-10ADvp, LC-10AD, LC-20AD
Detector: ultraviolet visible spectrophotometer (SPD-10AVvp, SPD-10A, SPD-10AV, SPD-20A)
Autosampler: SIL-20AC, SIL-10A, SIL-20ACHT
Column oven: CTO-10ACvp, CTO-10AC, CTO-20AC
System controller: SCL-10Avp, CBM-20^{a}
HPLC measurement conditions
Detector: ultraviolet absorption spectrophotometer (measurement wavelength: 240 nm)
Column: commercially available column (TSK-GEL Octyl-80TS, manufactured by Tosoh Corporation) which is a stainless tube (inner diameter 4.6 mm, length 150 mm) filled with 5 μm of octylsilyl silica gel for liquid chromatography
Column temperature: constant temperature near 40° C.
Mobile phase: acetonitrile/water mixed solution (1:1) flow rate: Adjusted to set the retention time of difluprednate to about 8 min.

### 3. Osmotic Pressure

A sample (0.2-0.3 mL) was placed in an osmometer cell, and the osmotic pressure was measured by an Advanced osmometer (Model 3900, ADVANCED INSTRUMENTS, INC.).

### Results

The residual ratio of tobramycin and difluprednate and osmotic pressure are shown in Table 2.

TABLE 2
Residual ratio of tobramycin and difluprednate after
storage at 60° C. for 2 weeks and osmotic pressure

| | Ex. | Ex. | Ex. | Comp. | Comp. | Comp. | Comp. | Comp. |
|---|---|---|---|---|---|---|---|---|
| formulation | 1 | 2 | 3 | Ex. 1 | Ex. 2 | Ex. 3 | Ex. 4 | Ex. 5 |
| tobramycin | 94.2 | 95.2 | 94.7 | 90.8 | 92.2 | 92.1 | 99.9 | 90.1 |
| residual | | | | | | | | |
| ratio (%) | | | | | | | | |
| difluprednate | 98.3 | 97.9 | 99.1 | — | — | — | — | — |
| residual | | | | | | | | |
| ratio (%) | | | | | | | | |
| osmotic pressure | 46 | 270 | 272 | — | — | — | — | — |
| (mOsm) | | | | | | | | |

While tobramycin was stable in the acetate buffer of Comparative Example 4, the residual ratio of tobramycin decreased by about 10% in the aqueous solution of Comparative Example 1 containing polysorbate 80, and the suspension of Comparative Example 5 which was Comparative Example 1 added with difluprednate after storage at 60° C. for 2 weeks. The results show that the stability of tobramycin decreases by the addition of polysorbate 80 to the liquid. On the other hand, a comparison of the residual ratio of tobramycin after storage at 60⁰C for 2 weeks between the aqueous solution of Comparative Example 1 and the emulsion of Example 1 reveals an improvement of the stability of tobramycin by 3.4% in Example 1. The results show that tobramycin is stabilized in the dosage form of an emulsion in a liquid in the presence of polysorbate 80 as compared to that in an aqueous solution. Moreover, they show that, even when sodium edetate and boric acid are added to an aqueous solution of tobramycin (Comparative Example 2 and Example 2), and when concentrated glycerol is added to an aqueous solution of tobramycin (Comparative Example 3 and Example 3), tobramycin is stabilized in the dosage form of an emulsion in a liquid in the presence of polysorbate 80 as compared to that in an aqueous solution.

### Experimental Example 2

In Experimental Example 1, it was clarified that the tobramycin content decreased in an aqueous solution and suspension added with polysorbate 80. Thus, whether or not tyloxapol, polyoxyethylene hydrogenated castor oil 60 (HCO-60) and polyoxyl 40 stearate (MYS-40), which are non-ionic surfactants widely used as additives for pharmaceutical products like polysorbate 80, influence the stability of tobramycin was evaluated. In addition, the storage stability of tobramycin in the presence of a non-ionic surfactant when the dosage form was changed from aqueous solution to emulsion was evaluated. In addition, an influence of the addition of boric acid on the stability of tobramycin was confirmed.

TABLE 3
Formulations of Examples 4 to 9 and Comparative Examples 6 to 8

| component | | Ex. | Ex. | Ex. | Ex. | Ex. | Ex. | Comp. | Comp. | Comp. |
|---|---|---|---|---|---|---|---|---|---|---|
| (g/100 mL) | grade | 4 | 5 | 6 | 7 | 8 | 9 | Ex. 6 | Ex. 7 | Ex. 8 |
| difluprednate | JPC | 0.05 | 0.05 | 0.05 | 0.05 | 0.05 | 0.05 | — | — | — |
| tobramycin | EP | 0.3 | 0.3 | 0.3 | 0.3 | 0.3 | 0.3 | 0.3 | 0.3 | 0.3 |
| castor oil | JP | 5.0 | 5.0 | 5.0 | 5.0 | 5.0 | 5.0 | — | — | — |
| tyloxapol | USP | 4.0 | 4.0 | — | — | — | — | 4.0 | — | — |
| HCO-60 | reagent | — | — | 4.0 | 4.0 | — | — | — | 4.0 | — |
| MYS-40 | reagent | — | — | — | — | 4.0 | 4.0 | — | — | 4.0 |
| boric acid | JP | — | 1.3 | — | 1.3 | — | 1.3 | — | — | — |
| sodium acetate | JP | 0.05 | 0.05 | 0.05 | 0.05 | 0.05 | 0.05 | 0.05 | 0.05 | 0.05 |
| sodium hydroxide/ | JP | q.s. | q.s. | q.s. | q.s. | q.s. | q.s. | q.s. | q.s. | q.s. |
| hydrochloric acid | | | | | | | | | | |
| purified water | — | q.s. | q.s. | q.s. | q.s. | q.s. | q.s. | q.s. | q.s. | q.s. |
| (total amount 100 | | | | | | | | | | |
| mL) | | | | | | | | | | |
| pH | — | 5.5 | 5.5 | 5.5 | 5.5 | 5.5 | 5.5 | 5.5 | 5.5 | 5.5 |

Examples 4 to 9 are emulsions, and Comparative Examples 6 to 8 are aqueous solutions. USP means the United States Pharmacopeia.

### Preparation Method of Examples 4 to 9

1) Based on the above-mentioned formulation, any one surfactant from tyloxapol (manufactured by Ruger Chemical Co., Inc.), HCO-60 (manufactured by Nikko Chemicals Co., Ltd.) and MYS-40 (manufactured by Nikko Chemicals Co., Ltd.) and sodium acetate were dissolved in purified water.
2) Separately, difluprednate was added to castor oil, and dissolved in water bath (85-95° C.) (solution A).
3) Solution A was gradually added to the solution of 1) (65-75° C.) to allow coarse emulsification (stirrer: Robo Mics, TOKUSHU KIKA KOGYO, rotating speed: about 8000 rpm, stirring time: 1 hr after addition of solution A).
4) After allowing to cool to room temperature, hydrochloric acid was added to adjust the pH to 5.4-5.6.
5) Purified water was added to dilute to 95% of a predetermined volume.
6) Using a high-pressure emulsifier (microfluidizer, Microfluidics Corporation), a micronized emulsion was prepared at treatment pressure about 1500 kgf/cm², water bath temperature for sample cooler about 35-45° C., number of passes 20 times.
7) Tobramycin and boric acid were added and dissolved in the micronized emulsion.
8) Hydrochloric acid was added to the micronized emulsion to adjust the pH to 5.4-5.6.
9) Purified water was added to the micronized emulsion to dilute to a predetermined volume.

### Preparation Method of Comparative Examples 6 to 8

1) Based on the above-mentioned formulation, any one surfactant from tyloxapol (manufactured by Ruger Chemical Co., Inc.), HCO-60 (manufactured by Nikko Chemicals Co., Ltd.) and MYS-40 (manufactured by Nikko Chemicals Co., Ltd.), sodium acetate and tobramycin were dissolved in purified water.
2) Hydrochloric acid was added to adjust the pH to 5.4-5.6.
3) Purified water was added to dilute to a predetermined volume.
   Test conditions

Examples 4 to 9 and Comparative Examples 6 to 8 prepared above were filled by 5 mL in 5 mL colorless glass ampoules and used as samples. The tobramycin content in the samples on preparation and after storage at 60° C. for 2 weeks was measured by the method described in Experimental Example 1. The residual ratio (%) of tobramycin content after storage at 60° C. for 2 weeks to that on preparation as 100% was determined. In Examples 4 to 9, the difluprednate content of the samples on preparation and after storage at 60° C. for 2 weeks was measured by the method described in Experimental Example 1, and the residual ratio (%) after storage at 60° C. for 2 weeks was determined. In Examples 4 to 9, moreover, the osmotic pressure on preparation was measured by the method described in Experimental Example 1.

### Results

The residual ratio of tobramycin and difluprednate and osmotic pressure are shown in Table 4.

TABLE 4
Residual ratio of tobramycin and difluprednate after
storage at 60° C. for 2 weeks and osmotic pressure

| | Ex. | Ex. | Ex. | Ex. | Ex. | Ex. | Comp. | Comp. | Comp. |
|---|---|---|---|---|---|---|---|---|---|
| formulation | 4 | 5 | 6 | 7 | 8 | 9 | Ex. 6 | Ex. 7 | Ex. 8 |
| tobramycin | 96.3 | 96.7 | 94.1 | 94.1 | 93.5 | 92.5 | 93.1 | 85.0 | 86.6 |
| residual | | | | | | | | | |
| ratio (%) | | | | | | | | | |
| difluprednate | 97.8 | 96.8 | 98.1 | 98.1 | 96.9 | 97.5 | — | — | — |
| residual | | | | | | | | | |
| ratio (%) | | | | | | | | | |
| osmotic pressure | 38 | 273 | 42 | 277 | 48 | 281 | — | — | — |
| (mOsm) | | | | | | | | | |

The residual ratio of tobramycin in an aqueous solution containing tyloxapol, polyoxyethylene hydrogenated castor oil 60 (HCO-60) or polyoxyl 40 stearate (MYS-40) decreased as in the case in an aqueous solution containing polysorbate 80. Comparisons of Comparative Example 6 and Example 4, Comparative Example 7 and Example 6, and Comparative Example 8 and Example 8 reveal that, by employing a dosage form of emulsion, the stability of tobramycin was improved as in the case using polysorbate 80, even when polysorbate 80 was changed to tyloxapol, polyoxyethylene hydrogenated castor oil 60 (HCO-60) or polyoxyl 40 stearate (MYS-40). Moreover, comparisons of Example 4 and Example 5, Example 6 and Example 7, and Example 8 and Example 9 reveal that the tobramycin content did not decrease remarkably even when boric acid was added to an emulsion containing tobramycin, and an isotonic tobramycin-containing emulsion could be prepared.

### Formulation Example

TABLE 5
Example of formulation containing difluprednate and tobramycin

| | | Formulation |
|---|---|---|
| component (g/100 mL) | grade | Example 1 |
| difluprednate | JPC | 0.05 |
| tobramycin | EP | 0.3 |
| castor oil | JP | 5.0 |
| polysorbate 80 | JP | 4.0 |
| sodium acetate | JP | 0.05 |
| sodium chloride | JP | 0.6 |
| sodium edetate | JP | 0.02 |
| sodium hydroxide/hydrochloric acid | JP | q.s. |
| purified water (total amount 100 mL) | — | q.s. |
| pH | — | 5.5 |

| | |
|---|---|
| tobramycin residual ratio (%) after storage at | 98.6 |
| 60° C. for 2 weeks | |
| difluprednate residual ratio (%) after storage at | 98.6 |
| 60° C. for 2 weeks | |
| osmotic pressure (mOsm) | 274 |

### Preparation Method of Formulation Example 1

1) Based on the above-mentioned formulation, polysorbate 80, sodium acetate, sodium edentate and tobramycin were dissolved in purified water.
2) Hydrochloric acid was added to adjust the pH to 5.4-5.6.
3) Separately, difluprednate was added to castor oil, and dissolved in a water bath (85-95° C.) (solution A).
4) Solution A was gradually added to the solution of 2) (65-75° C.) to allow coarse emulsification (stirrer: Robo Mics, TOKUSHU KIKA KOGYO, rotating speed: about 8000 rpm, stirring time: 1 hr after addition of solution A).
5) After allowing to cool to room temperature, pH was confirmed (pH 5.4-5.6).
6) Purified water was added to dilute to a predetermined volume.
7) Using a high-pressure emulsifier (microfluidizer, Microfluidics Corporation), a micronized emulsion was prepared at treatment pressure about 1500 kgf/cm², water bath temperature for sample cooler about 35-45° C., number of passes 20 times.
8) Sodium chloride was dissolved in the micronized emulsion, and hydrochloric acid was added to adjust the pH to 5.4-5.6.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the spirit and scope of the appended claims.

The contents disclosed in any publication cited herein, including patents and patent applications, are hereby incorporated in their entireties by reference, to the extent that they have been disclosed herein.

This application is based on U.S. provisional patent application No. 61/417,651, the contents of which are entirely incorporated hereinto.

1. An oil-in-water emulsion composition for topical administration, comprising (a) tobramycin, (b) difluprednate, (c) water, (d) oil and (e) an emulsifier.

2. The composition according to claim 1, wherein said oil is selected from the group consisting of castor oil, peanut oil, cottonseed oil, soybean oil, olive oil and medium-chain triglyceride.

3. The composition according to claim 2, wherein said oil is castor oil.

4. The composition according to claim 1, wherein said emulsifier is a non-ionic surfactant.

5. The composition according to claim 4, wherein said non-ionic surfactant is at least one selected from the group consisting of polysorbate 80, tyloxapol, polyoxyethylene hydrogenated castor oil 60 and polyoxyl 40 stearate.

6. The composition according to claim 1, which is an eye drop, a nasal drop, an ear drop or a lotion.

7. The composition according to claim 1, which comprises 0.01-0.2 (w/v)% of difluprednate, 0.1-1 (w/v)% of tobramycin, 1-20 (w/v)% of castor oil and 1-20 (w/v)% of polysorbate 80, and has a pH of 4-7.

8. A method for stabilizing tobramycin, comprising mixing (a) tobramycin, (b) difluprednate, (c) water, (d) oil and (e) an emulsifier to form an oil-in-water emulsion.
